SEQUENCE LISTING (6 sequences)

SEQ ID NO: 1 - DNA, 1752 bp, mouse
atgagacaggacatgccaaagccctcagaggcagcgcgttgctgctctggcctggccaggcgagcactcacgatcatctttgccctgctcatcctgggcctcatgacctgggcctacgccgcaggcgttcctctggcttcagatcgctatggactcctggcctttggcctctatggggcattcctcagcgcacacctagtggcacagagcctcttcgcttacctggagcaccgaagggtggcagcggctgcgcggcgctccttggcgaaggggcccctggatgcggccactgcacgcagcgtggcactcaccatctcagcctaccaagaggatcccgcttacctgcgccagtgcttgacctccgcgcgcgccttgctgtacccgcacacgaggttacgcgtgctcatggtggtggacggcaaccgcgctgaggatctgtacatggtggacatgttccgagaagtcttcgccgatgaggaccccgccacttatgtgtgggatggcaactaccatcagccctgggaaccagcggaggctacgggcgctgtcggtgaaggtgcctaccgggaggtggaggcggaggaccccgggcggttggcggtggaggcgctggtgagaacacgcaggtgcgtgtgcgtggctcagcgttggggcggcaaacgtgaggtcatgtacacagctttcaaggcactgggcgactccgtggactacgtgcaggtctgtgactcagacacaagactagaccccatggcactgctggagcttgtgcgagtgttggatgaagacccccgggtaggggctgttggaggggatgtgaggatccttaaccctctggactcctgggtcagcttcttgagcagtcttcgatactgggtagccttcaatgtggaacgagcttgtcagagctacttccactgtgtgtcctgcatcagtggtcctctgggtctatacagaaacaatctcctgcagcagttcttggaggcctggtacaaccaaaagttcctgggcacccactgcacatttggggatgacaggcacctcaccaaccgaatgcttagcatgggctatgctaccaagtatacctcgcgctccagatgctactcggagacgccctcctccttccttcgttggttgagccaacagacccgctggtccaaatcttacttccgagagtggctatacaatgctctgtggtggcatcgccaccacgcatggatgacctatgaagcggtggtctcgggcctcttccctttcttcgtggctgccacggtgttgaggctcttctatgcagggcgcccgtgggctctgctctgggtgctgctctgtgtgcagggcgtagcactggcaaaggcagcctttgcagcctggctgcgtggctgcgtgcgcatggtgctgctgtcactctatgcaccactctacatgtgcggcctcctgcctgccaaattcctagcgttggttaccatgaatcaaagtggttggggtacctcgggccggaagaaactggctgctaactatgtccccgtgttgcccctggcactctgggctctactgctgcttggaggcctggcccgcagtgtggcccaggaggccagagctgactggagtggcccatcccgagcagctgaagcctaccaccttgctgctggggctggtgcctatgtggcctactgggtggtaatgttaactatctactgggtaggtgtgaggaggctgtgcagacgtcggagcggtggttaccgtgtccaagtatga

SEQ ID NO: 2 - DNA, 1659 bp, mouse
atgcattgtgagaggtttctatgtgtcctgagaataattggaactacactttttggagtgtctctcctcctcggaatcacagctgcttatattgttggctaccagtttatccaaacagataattactacttctcatttggactgtacggtgcctttttagcctcgcatctcatcatccaaagcctctttgcctttttggaacaccggaaaatgaagaagtcccttgaaaccccgattaaattgaacaaaacggtagcactctgcatcgctgcgtaccaagaggaccctgactacttacggaaatgtttgcaatctgtgaaaaggctgacctaccctgggattaaagtcgtgatggtcatcgatgggaactcagacgacgacctttacatgatggacatattcagcgaagttattggcagggacaaatcggccacgtacatctggaagaacaactttcatgaaaagggacctggtgagacagaagagtcccataaagaaagttcacaacatgtcacccaattggtcttgtctaacaaaagtatttgcatcatgcaaaaatggggtggaaagagagaagtcatgtacacagccttcagagcactggggcgaagcgtggattatgtacaggtgtgtgactcagatactatgcttgaccctgcctcatctgtggagatggtgaaggtcttagaggaagaccctatggttggaggtgttggaggagatgtccagattttaaacaagtatgattcctggatctccttcctcagcagcgtgagatactggatggcttttaatatagaaagggcctgccagtcttattttggctgtgtccagtgcataagcggtcctctgggaatgtacagaaactccttgctgcatgaatttgtggaagactggtacaatcaggaattcatgggtaaccaatgcagttttggtgacgacaggcaccttaccaacagggtgttgagtctgggctatgcaactaaatacacggctcggtccaagtgccttactgaaactcccatagaatatctgagatggctgaaccagcagacccgatggagcaagtcctacttccgagagtggctgtacaatgccatgtggtttcacaagcatcacctgtggatgacctatgaagctgttatcactggattctttcctttctttctcattgccacagtcatccagctcttctacaggggtaaaatctggaacatcctcctcttcctgttaactgtccagctagtgggtctcatcaagtcatcttttgccagctgccttagaggaaatatcgtcatggtattcatgtctctgtattcagtgttatacatgtcaagtctacttcctgccaagatgtttgcaattgcaaccataaacaaagctgggtggggcacatctggaaggaagaccattgttgttaatttcataggacttattccagtgtccgtgtggtttacaatccttctaggtggtgtaattttcaccatttataaggaatctaaaaagccattttccgaatccaaacagactgttctcatcgtgggaactttgatctatgcatgctactgggtcatgcttttgactctctatgtggttctcatcaataagtgtggcaggcggaagaagggacaacagtatgacatggtgcttgatgtatga

SEQ ID NO: 3 - DNA, 1665 bp, mouse
atgccggtgcagctgactacagccctgcgtgtggtgggcaccagtctgtttgccctggtagtgctgggaggcatcctggcggcctatgtgacaggctaccagtttatccacacagaaaagcactacctgtcctttggcctctacggtgccatcctgggtctacatctgctcatccagagcctgtttgccttcctggagcaccgtcgaatgcgcagggcagggcgccccctcaagctgcactgctcccagaggtcgcgttcagtggcactctgcattgctgcctaccaagaggaccccgaatacctgcgcaagtgccttcgctcagctcagcgcattgcctttccaaacctcaaggtggtcatggtagtggatggcaatcgccaggaagatacctacatgttggacatcttccatgaggtgctgggtggcactgagcaagctggcttctttgtgtggcgtagcaatttccatgaggcgggtgaaggagagacagaggccagcctgcaggaaggcatggagcgtgtgcgagctgtggtgtgggccagcaccttctcatgcatcatgcagaagtgggggggcaagcgtgaggtcatgtacactgccttcaaggcccttggcaactcagtggactacatccaggtgtgtgactctgacactgtgctggacccagcctgcaccattgagatgcttcgagtcttggaagaagatccccaagtaggaggtgttggaggagatgtccaaatcctcaacaagtatgattcatggatctccttcctgagcagtgtgaggtactggatggctttcaacgtggagcgggcctgccagtcctactttggctgtgtgcaatgtattagtgggcctttgggcatgtaccgcaacagcctccttcagcagttcctggaggattggtaccatcagaagttcctaggcagcaagtgcagctttggggatgatcggcaccttaccaaccgagtcctgagtcttggctaccggactaagtatacagcacgctctaagtgcctcacagagacccccactaggtaccttcgatggctcaatcagcaaacccgctggagcaagtcttactttcgggaatggctctacaattctctgtggttccataagcaccacctctggatgacctatgaatcagtggtcacaggtttcttcccattcttcctcattgctacagtcatacaacttttctaccgtggccgcatctggaacattctcctcttcctgctaacagtgcagctggtgggcattatcaaggctacctatgcctgcttccttcgaggcaatgcagagatgatcttcatgtccctctactcccttctctatatgtccagcctcttgccagccaagatctttgctattgctaccatcaacaagtctggctggggcacttctggcaggaaaaccattgtcgtgaacttcattggcctaatccccgtgtccatctgggtggcagttcttctaggggggttagcctacacagcttattgccaggacctgttcagtgagaccgagctagccttcctagtctctggggccatcctgtatggctgctactgggtggccctcctcatgctgtatctggccattattgcccggaggtgtgggaagaagccagaacagtatagcctggcttttgcggaggtgtga

SEQ ID NO: 4 - protein, 583 aa, mouse
MRQDMPKPSEAARCCSGLARRALTIIFALLILGLMTWAYAAGVPLASDRYGLLAFGLYGAFLSAHLVAQSLFAYLEHRRVAAAARRSLAKGPLDAATARSVALTISAYQEDPAYLRQCLTSARALLYPHTRLRVLMVVDGNRAEDLYMVDMFREVFADEDPATYVWDGNYHQPWEPAEATGAVGEGAYREVEAEDPGRLAVEALVRTRRCVCVAQRWGGKREVMYTAFKALGDSVDYVQVCDSDTRLDPMALLELVRVLDEDPRVGAVGGDVRILNPLDSWVSFLSSLRYWVAFNVERACQSYFHCVSCISGPLGLYRNNLLQQFLEAWYNQKFLGTHCTFGDDRHLTNRMLSMGYATKYTSRSRCYSETPSSFLRWLSQQTRWSKSYFREWLYNALWWHRHHAWMTYEAVVSGLFPFFVAATVLRLFYAGRPWALLWVLLCVQGVALAKAAFAAWLRGCVRMVLLSLYAPLYMCGLLPAKFLALVTMNQSGWGTSGRKKLAANYVPVLPLALWALLLLGGLARSVAQEARADWSGPSRAAEAYHLAAGAGAYVAYWVVMLTIYWVGVRRLCRRRSGGYRVQV

SEQ ID NO: 5 - protein, 552 aa, mouse
MHCERFLCVLRIIGTTLFGVSLLLGITAAYIVGYQFIQTDNYYFSFGLYGAFLASHLIIQSLFAFLEHRKMKKSLETPIKLNKTVALCIAAYQEDPDYLRKCLQSVKRLTYPGIKVVMVIDGNSDDDLYMMDIFSEVIGRDKSATYIWKNNFHEKGPGETEESHKESSQHVTQLVLSNKSICIMQKWGGKREVMYTAFRALGRSVDYVQVCDSDTMLDPASSVEMVKVLEEDPMVGGVGGDVQILNKYDSWISFLSSVRYWMAFNIERACQSYFGCVQCISGPLGMYRNSLLHEFVEDWYNQEFMGNQCSFGDDRHLTNRVLSLGYATKYTARSKCLTETPIEYLRWLNQQTRWSKSYFREWLYNAMWFHKHHLWMTYEAVITGFFPFFLIATVIQLFYRGKIWNILLFLLTVQLVGLIKSSFASCLRGNIVMVFMSLYSVLYMSSLLPAKMFAIATINKAGWGTSGRKTIVVNFIGLIPVSVWFTILLGGVIFTIYKESKKPFSESKQTVLIVGTLIYACYWVMLLTLYVVLINKCGRRKKGQQYDMVLDV

SEQ ID NO: 6 - protein, 554 aa, mouse
MPVQLTTALRVVGTSLFALVVLGGILAAYVTGYQFIHTEKHYLSFGLYGAILGLHLLIQSLFAFLEHRRMRRAGRPLKLHCSQRSRSVALCIAAYQEDPEYLRKCLRSAQRIAFPNLKVVMVVDGNRQEDTYMLDIFHEVLGGTEQAGFFVWRSNFHEAGEGETEASLQEGMERVRAVVWASTFSCIMQKWGGKREVMYTAFKALGNSVDYIQVCDSDTVLDPACTIEMLRVLEEDPQVGGVGGDVQILNKYDSWISFLSSVRYWMAFNVERACQSYFGCVQCISGPLGMYRNSLLQQFLEDWYHQKFLGSKCSFGDDRHLTNRVLSLGYRTKYTARSKCLTETPTRYLRWLNQQTRWSKSYFREWLYNSLWFHKHHLWMTYESVVTGFFPFFLIATVIQLFYRGRIWNILLFLLTVQLVGIIKATYACFLRGNAEMIFMSLYSLLYMSSLLPAKIFAIATINKSGWGTSGRKTIVVNFIGLIPVSIWVAVLLGGLAYTAYCQDLFSETELAFLVSGAILYGCYWVALLMLYLAIIARRCGKKPEQYSLAFAEV

(19) United States
(12) Patent Application Publication
(10) Pub. No.: US 2003/0087850 A1
(43) Pub. Date: May 8, 2003

(54) Gene therapy for dry eye syndrome

(76) Inventors: Philip DeHazya, Westbury, NY (US); Weiliam Chen, Coram, NY (US)

Correspondence Address: DARBY & DARBY P.C., P. O. BOX 5257, NEW YORK, NY, 10150-5257, US

(21) Appl. No.: 09/902,939

(22) Filed: Jul. 10, 2001

(51) Int. Cl. A61K 48/00; C07H 21/04; C08B 37/00

(52) U.S. Cl. 514/44; 514/54; 536/23.1; 536/53

(57) ABSTRACT

The present invention provides bioconjugates comprising substances which provide increased safety and bioavailability of nucleic acids when used in gene therapy applications. The dihydrazide derivatized hyaluronic acid/nucleic acid compositions of the present invention include hyaluronic acid which has been derivatized with a dihydrazide, preferably adipic dihydrazide, which is crosslinked to a nucleic acid. These compositions may be included in microsphere, film, wafer, matrix, hydrogel, gel and sol formulations. These bioconjugates are useful in gene therapy applications for the treatment of a variety of medical conditions including dry eye syndrome or other medical conditions wherein an increase in the production of hyaluronic acid in the eye would be therapeutic. Further, there may be other medical conditions which could benefit from increased production of hyaluronan, such as osteoarthritis of the articular joints. In the treatment of dry eye syndrome, the compositions of the invention include a hyaluronan synthase gene. When cells in the eye are transfected with and express hyaluronan synthase, hyaluronic acid production is a result. Hyaluronic acid production in the eye provides a therapeutic effect in the treatment of dry eye syndrome by augmenting production of or composition of the ocular tear film. Production of the compositions of the invention is also possible in a process which is easily modified for production of large quantities of dihydrazide derivatized hyaluronic acid.

### FIELD OF THE INVENTION

The present invention relates to the use of derivatized hyaluronic acid/nucleic acid compositions, preferably dihydrazide derivatized hyaluronic acid/nucleic acid compositions, and microsphere, film, wafer, matrix, gel and sol formulations comprising these compositions. The invention also relates to the use of the compositions of the invention, preferably, wherein the nucleic acid is a hyaluronan synthase (HAS) gene, in gene therapy applications to treat dry eye syndrome(DES). Methods for the production of these compositions are also provided.

### BACKGROUND OF THE INVENTION

Dry eye is a chronic condition characterized, simplistically, by a paucity of moisture in the eye. The cause of dry eye can be infectious disease, the aging process, autoimmunity, trauma or other pathological changes. There is no single cause. Symptoms of dry eye include scarcity of moisture and lubrication or tear film, itching, burning, inflammation, erythema, distortion of vision and in severe cases damage to the tissue of the eye leading to blindness.

The tear film of the eye protects ocular tissues by providing moisture and lubrication. It is composed of three major components: water (whose primary source is the lachrymal duct), lipid and “mucin”, a proteoglycan mixture containing polymeric forms of carbohydrate and protein. The tear film of the eye is compromised in most patients with dry eye, in that the water component is reduced in quantity. The tear volume may be determined by the schirmer test. This is a test using a filter paper strip with graduations. The strip is placed at the lower lid and the tear is allowed to wick into the paper for a set time interval. The more liquid, the farther the liquid edge travels. The distance the tear fluid travels determines whether the person is determined to have dry eye syndrome. These tear film alterations lead to the symptoms observed in DES.

Dry eye syndrome is a common, chronic condition. It is defined and clinically diagnosed based on patient history and actual assessment of the amount of tear film (volume) in and around the eye. The development of DES is correlated with a variety of conditions, diseases, pathologies and etiologies. Infection, Sjogrens syndrome, trauma, myobium disease, and lacrymal gland dysfunction can all result in dry eye. Thus, there is not one, but many causes, eliminating the possibility of providing a single missing function or correcting a defined genetic defect, as some gene therapy approaches seek to do.

It is estimated that about 20 million, mostly older, Americans and about twice this number world-wide, experience dry eye symptoms. Current treatment of dry eye usually consists of instillation of drops or “artificial tears” of prescription or over the counter formulations. The topical application of hyaluronic acid (HA) to the eyes in the treatment of dry eye syndrome has been reported previously. He, Z., et al., in Yen Ko Hseuh Pao, 6: 111-2, 1990 reports the human clinical testing of an HA-containing eye drop for dry eye syndrome. Shimmura, S., et al., Br. J. Ophthamol., 79: 1007-11, 1995 report on rabbit testing of HA-containing dry eye drops. Another publication concerning human clinical testing of HA-containing dry eye drops is by Solomon, A., et al, J. Ocul. Pharmacol. Ther. 14 (6): 497-504, 1998. While some topical treatments contain hyaluronic acid in dilute solution, others contain physiologically acceptable salt solutions. Their major function is to provide moisture and lubrication. Their application numerous times per day is usual. Ointments can also be used for this purpose. More severe cases of dry eye can be treated by installing punctal plugs to slow or reduce the drainage of tear liquid out of the eye, by balloon catherization of lacrymal ducts in an attempt to open them, and by surgery. None of these treatments is permanent, they are inconvenient and bothersome and are only somewhat effective.

Recently, several dry eye products have been formulated to contain hyaluronic acid. Hyaluronic acid has been shown to be anti-inflammatory and to stimulate the growth of corneal epithelial cells in in vitro and in in vivo tests. HA is also lubricious and hygroscopic. Although these products show some advantages over older and more conventional formulations, they must be applied several times per day and do not relieve the symptoms of dry eye for more than a short period of time. Thus, there is still a need for an efficacious, more convenient, longer term remedy for dry eye.

Hyaluronan synthase (HA synthase) is the enzyme that synthesizes hyaluronic acid in vertebrate cells. The enzyme is found in the cell membrane and extrudes polymerized sugar precursors from within the cell to the external milieu. There are 3 known forms or isotypes of HA synthase (HAS1, HAS2, and HAS3) that differ in catalytic activity and in the type of hyaluronan they synthesize. Although three types of HAS have been characterized, there are likely to be several other physiologically relevant isoforms of the enzyme. Cells expressing transfected forms of some HA synthases form coats around themselves, suggesting a protective function of HA.

Because HA is a component of the tear film, and has anti-inflammatory and lubricating properties, enhancement of HA secretion in the eye of dry eye patients is of clinical benefit. One way to enhance HA secretion is by transfection of the tissues of the eye with HA synthase genes. Expression of the genes in the ocular tissues will increase the HA content of the tear film. This provides relief of the symptoms of dry eye which is longer-lived than that provided by therapies which are currently available.

HAS1-3 Homologues

The HAS1-3 genes encode a family of proteins which are highly conserved in several, diverse animal species. The following is a summary of the HAS1-3 homologues which are known in the art. The numbers represent the percentage of nucleotide/amino acid identity or homology shared between the mouse HAS sequences set forth in SEQ ID NOs. 1-6 and the specified homologues.

SEQ ID NO. 1=Mouse HAS1 DNA

SEQ ID NO. 2=Mouse HAS2 DNA

SEQ ID NO. 3=Mouse HAS3 DNA

SEQ ID NO. 4=Mouse HAS1 Protein

SEQ ID NO. 5=Mouse HAS2 Protein

SEQ ID NO. 6=Mouse HAS3 Protein

TABLE 1
Percent sequence identity between SEQ ID NOs. 1-3 and
several HAS genes.

| | SEQ ID NO. | SEQ ID NO. | SEQ ID NO. |
|---|---|---|---|
| HAS Gene | 1 | 2 | 3 |
| Human HAS1 | 85 | — | — |
| Bos taurus HAS1 | 88 | — | — |
| Mus musculus HAS1 | 100 | — | — |
| Mus musculus HAS2 | — | 99 | — |
| Rattus norvegicus HAS2 | — | 95 | — |
| Bos taurus HAS2 | — | 89 | — |
| Gallus gallus HAS2 | — | 84 | — |
| Xenopus laevis HAS2 | — | 81 | — |
| Human HAS2 | — | 90 | — |
| Mus musculus HAS3 | — | — | 99 |
| Human HAS3 | — | — | 90 |
| Bos taurus HAS3 | — | — | 91 |
| Gallus gallus HAS3 | — | — | 83 |
| Xenopus laevis HAS3 | — | — | 79 |

TABLE 2
Percent sequence identity or homology between SEQ ID NOs. 4-6
and several HAS proteins*

| | SEQ ID NO. | SEQ ID NO. | SEQ ID NO. |
|---|---|---|---|
| HAS Protein | 4 | 5 | 6 |
| Human HAS1 | 84, 85 | — | — |
| Xenopus laevis HAS1 | 52, 66 | — | — |
| (DG42) | | | |
| Bos taurus HAS1 | 94, 94 | — | — |
| Mus musculus HAS1 | 87, 87 | — | — |
| Mus musculus HAS2 | — | 95, 95 | — |
| Human HAS2 | — | 94, 95 | — |
| Bos taurus HAS2 | — | 93, 94 | — |
| Rattus norvegicus HAS2 | — | 94, 94 | — |
| Gallus gallus HAS2 | — | 88, 92 | — |
| Xenopus laevis HAS2 | — | 83, 90 | — |
| Mus mucularis HAS3 | — | — | 96, 96 |
| Human HAS3 | — | — | 93, 94 |
| Gallus gallus HAS3 | — | — | 88, 91 |
| Xenopus laevis HAS3 | — | — | 85, 89 |
| Bos taurus HAS3 | — | — | 91, 91 |

*The number on the left indicates the level of sequence identity between the specified sequences and the number to the right specifies the level of sequence homology between the specified sequences.

The foregoing sequence analysis was performed by comparing SEQ ID NOs. 1-6 to the sequences in the Genbank database using a BLASTN (Table 1) or BLASTP (Table 2) algorithm. BLAST algorithms are discussed in detail infra.

### SUMMARY OF THE INVENTION

The present invention relates to a method for transfecting a cell (in cell culture or in vivo), preferably an ocular cell, with a nucleic acid (e.g., plasmid DNA), preferably hyaluronan synthase (e.g, HAS1-3) which comprises hyaluronan synthase activity, more preferably wherein said nucleic acid has a nucleotide sequence of about 70% to about 100% identity to a reference nucleotide sequence selected from the group consisting of SEQ ID NOs. 1-3 or wherein the nucleic acid has a nucleotide sequence which encodes a hyaluronan synthase enzyme (e.g, HAS1-3), preferably the amino acid sequence of the enzyme comprises about 70% to about 100% homology or identity to a reference amino acid sequence selected from the group consisting of SEQ ID NOs.4-6 wherein homology is determined using the BLASTN or BLASTP algorithm, respectively, where the parameters of the algorithm are selected to give the largest match between the sequences tested over the entire length of the reference sequence; including the step of contacting the cell with a derivatized hyaluronic acid/nucleic acid bioconjugate which comprises said nucleic acid or, preferably, with a dihydrazide derivatized hyaluronic acid/nucleic acid bioconjugate comprising hyaluronic acid crosslinked with adipic dihydrazide wherein the adipic dihydrazide is further crosslinked to the nucleic acid. Preferably, the method is used in the treatment of dry eye syndrome. The present invention also includes the above-described bioconjugates.

The invention also includes a method of producing a dihydrazide derivatized hyaluronic acid/nucleic acid bioconjugate comprising;

(a) contacting hyaluronic acid with (i) a nucleic acid comprising a hyaluronan synthase gene, preferably wherein the nucleic acid comprises a nucleotide sequence of at least about 70% to about 100% identity to a reference nucleotide sequence selected from the group consisting of SEQ ID NO: 1-3, wherein identity is determined using the BLASTN algorithm, where the parameters are selected to give the largest match between the sequences tested, over the entire length of the selected reference sequence or with (ii) a nucleic acid encoding a hyaluronan synthase protein comprising an amino acid sequence which has about 70% to about 100% homology or identity to a reference amino acid sequence selected from the group consisting of SEQ ID NOs. 4-6 wherein homology or identity is determined using a BLASTP algorithm where the parameters of the algorithm are selected to give the largest match between the sequences tested over the entire length of the selected reference sequence;

(b) drying the mixture of hyaluronic acid and the nucleic acid;

(c) suspending the dried material in a solution of 90% dimethyl formamide containing a dihydrazide cross-linker and a carbodimide;

(d) adjusting the pH of the suspension of step (c) to the acidic range and, optionally, incubating the mixture for a period of time (e.g., 6 hours, 24 hours, or 48 hours);

(e) isolating the suspended material from the suspension of step (d); and

(f) washing the isolated material from step (e) with an alcohol.

Methods of modulating the extent of crosslinking are also provided. The extent of cross linking may be increased by increasing the incubation period of step (d) or decreased by decreasing the incubation period of step (d). The extent of crosslinking may also be increased by further decreasing the pH at step (d) or decreased by raising the pH at step (d). Increasing the concentration of crosslinker at step (c) will increase the extent of crosslinking whereas decreasing the concentration will decrease the extent of crosslinking.

In one embodiment of the invention, the above steps are performed in the order a, b, c, d, e, and f. However, the order in which the steps are conducted is not a critical feature of the method. In one embodiment, lyophilization is performed after cross linking in step (d).

### BRIEF DESCRIPTION OF THE FIGURES

FIG. 1. A representative HAS construct containing the HAS2 gene, a CMV promoter, antibiotic resistance elements, a poly adenylation site, a viral origin of DNA replication, and a peptide tag (poly histadine) that will allow the specific detection of the HAS protein derived from this plasmid, immunologically.

FIG. 2. The expression of green fluorescent protein (GFP) in the conjunctiva of living rat eyes.

FIG. 3. The expression of green fluorescent protein in the conjunctiva of living rat eyes.

FIG. 4. The expression of green fluorescent protein in the conjunctiva of living rat eyes (higher magnification).

FIG. 5. Schematic diagram of formulation steps for HA-DNA films and matrices.

FIG. 6. Release of DNA from 2 different formulations of HA-DNA micropheres. Microspheres were made using an emulsion polymerization method by mixing HA solutions and DNA together with adipic dihydrazide, DAC (carbodiimide) and mineral oil. The mixture was emulsified by rapid stirring with an organic emulsifier (e.g., Span 60). The pH was lowered by the addition of HCl (e.g., 1 N) to around 3.0. Stirring was continued for two different time intervals (6 hrs for batch A or 16 hours for batch B). The polymerized microspheres were recovered as described in U.S. patent application 09/596,665, filed Jun. 19, 2000, now pending and U.S. patent application 09/596,548 filed Jun. 19, 2000, now pending which are herein incorporated by reference in their entireties, dried and stored in a dry atmosphere. For the assay, microspheres were weighed and resuspended in physiological phosphate saline buffer. Bovine testicular hyaluronidase was added (e.g., 1 unit/ml) as appropriate. At the indicated time intervals, samples of the mixture were obtained, and soluble materials (released DNA) were separated from insoluble (HA/microsphere associated DNA) materials by centrifugation. The DNA was quantified using a thiazole orange assay by measuring the fluorescence of DNA bound to the dye.

FIG. 7. Chemical structures of dihydrazides which may be used in the present invention.

FIG. 8. ADH cross-linked, lyophilized, HA-DNA matrices. Left: low power (2×) and right, higher power (10×).

FIG. 9. Three formulations of cross-linked HA/DNA matrices. The matrices were cross-linked for 6, 12 and 24 hours (NOs. 2,1 and 3, respectively) at ambient temperature (approx. 22° C.), then placed in physiological buffer containing 1 unit testicular hyaluronidase/ml. The vials were incubated at 37° C. for 13 days. Ethidium bromide was added to visualize the DNA and the vials were photographed under short wave (330 nm) UV light. Formulation 3 is virtually intact showing almost complete resistance to hyaluronidase degradation, while #2 is completely degraded, having released all of the DNA.

### DETAILED DESCRIPTION OF THE INVENTION

The invention relates to the introduction of hyaluronan synthase genes to tissues of the eye to relieve the symptoms of DES. Hyaluronan synthase is an enzyme that causes the synthesis of hyaluronic acid in vertebrate cells. Three isoforms of the enzyme have been identified and are the products of 3 distinct but related genes (HAS1, HAS2 and HAS3). In preferred embodiments the hyaluronan synthase genes (HAS 1-3) comprise a nucleotide sequence set forth in SEQ ID NOs. 1-3, respectively. The corresponding amino acid sequences of hyaluronan synthases (HAS 1-3) are set forth in SEQ ID NOs.4-6, respectively. For the purposes of the present application “hyaluronan synthase” refers to any enzyme or gene encoding an enzyme which comprises hyaluronan synthase activity. Preferably, hyaluronan synthase is derived from animals (e.g., Xenpous laevis, Bos taurus, Gallus gallus, Mus musculus or Rattus norvegicus.) more preferably humans or mice. A protein may comprise hyaluronan synthase activity if said activity is detected in any of the hyaluronan synthase assays which are well known in the art. Hyaluronan synthase assays are discussed below.

The HAS gene encodes the form of the enzyme that synthesizes HA and secretes it from the cell into the surrounding mileau. The invention uses hyaluronan synthase genes and specifies the dihydrazide mediated cross-linking of the genes to hyaluronic acid to form a bioconjugate. In these bioconjugates, the nucleic acid is crosslinked to the derivatized hyaluronic acid, preferably the hyaluronic acid is dihydrazide derivativized, more preferably the hyaluronic acid is derivatized with adipic dihydrazide. The bioconjugate allows for the sustained delivery of biologically active genes to animal tissue targets which are contacted with or brought into the proximity of the bioconjugates. Without being bound by theory, it is believed that the nucleic acid is gradually introduced due to slow break down of the hyaluronic acid and release of the DNA into the surrounding mileau. Once released from the biconjugates, the nucleic acid may be taken up by a target cell. The successful use of hyaluronic acid and dihydrazide crosslinking to deliver biologically active genes was a surprising and unexpected discovery. This is because of the fragile nature of purified, biologically active DNA and the necessity of subjecting the DNA to chemicals and conditions which could potentially damage or interfere with its structure, integrity of bases or ability to code for biologically active proteins. Successful cross-linking was also unexpected because a hydrazide reactive moiety on the DNA was not readily apparent. Also unexpected, was the retention of the biological activity of the DNA in situ, in the presence of various catabolic and degradative enzymes, that allowed sustained cell transformation for several days after application of the dosage forms. For the purposes of this application, a bioconjugate wherein a nucleic acid is crosslinked to derivatized hyaluronic acid may be referred to as a “derivatized hyaluronic acid/nucleic acid bioconjugate”, “cross-linked HA”, “DNA/HA bioconjugate” or “bioconjugate”. Preferably, the biconjugates of the invention comprise a “dihydrazide derivativized hyaluronic acid/nucleic acid bioconjugate”. These terms include all formulations of this composition including microspheres, films, wafers, matrices, gels, sols and others.

The term “patient” or “subject” refers to any organism, preferably an animal, more preferably a mammal and most preferably a human.

### MOLECULAR BIOLOGY

In accordance with the present invention there may be employed conventional molecular biology, microbiology, and recombinant DNA techniques within the skill of the art. Such techniques are explained fully in the literature. The following publications are incorporated by reference: e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook, et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel, et al. (eds.), Current Protocols in Molecular Biology, John Wiley & Sons, Inc. (1994).

A “DNA molecule”, “nucleic acid molecule” or “nucleic acid” refers to the phosphodiester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms or to any particular length. A more specific term, “oligonucleotide”, refers to a nucleic acid molecule of 20 bases in length, or less. Thus, these terms include double-stranded DNA found, inter alia, in linear (e.g., restriction fragments) or circular DNA molecules, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation.

A “DNA sequence” or “nucleotide sequence” is a series of nucleotide bases (also called “nucleotides”) in DNA and RNA, and means any chain of two or more nucleotides. A nucleotide sequence typically carries genetic information, including the information used by cellular machinery to make proteins. These terms include double or single stranded genomic DNA or cDNA, RNA, any synthetic and genetically manipulated nucleic acid, and both sense and anti-sense nucleic acids. This includes single- and double-stranded molecules, i.e., DNA-DNA, DNA-RNA and RNA-RNA hybrids, as well as “protein nucleic acids” (PNA) formed by conjugating bases to an amino acid backbone. This also includes nucleic acids containing modified bases, for example thio-uracil, thio-guanine and fluoro-uracil.

The term “protein” refers to any peptide or polypeptide containing two or more amino acids, modified amino acids, or amino acid derivatives. “Protein”, by way of example, and without excluding other types of proteins, includes enzymes (e.g., HAS 1-3) and structural proteins.

The term “heterologous” refers to a combination of elements not naturally occurring. For example, heterologous DNA refers to DNA not naturally located in the cell, or in a chromosomal site of the cell. Preferably, the heterologous DNA includes a gene foreign to the cell. A heterologous expression regulatory element is such an element operatively associated with a different gene than the one with which it is operatively associated in nature.

The “nucleic acids” and “nucleic acid molecules” herein may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′- non-coding regions, and the like. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Preferred embodiments comprise modification by derivatization with hyaluronic acid, a dihydrazide (e.g., adipic dihydrazide) and/or a carbodiimide. Nucleic acids may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The nucleic acids may be derivatized by formation of a methyl or ethyl phosphotriester or an alkyl phosphoramidate linkage. Furthermore, the nucleic acids herein may also be modified with a label capable of providing a detectable signal, either directly or indirectly. Exemplary labels include radioisotopes, fluorescent molecules, biotin, and the like.

The term “host cell” means any cell of any organism that is selected, modified, transformed, grown, or used or manipulated in any way, for the production of a substance by the cell, for example the expression by the cell of a gene or DNA sequence. In specific embodiments of the invention, a host ocular cell is transfected with a hyaluronan synthase gene (e.g., HAS1-3) which drives expression of hyaluronic acid in the cell.

Proteins are made in the host cell using instructions in DNA and RNA, according to the genetic code. Generally, a DNA sequence having instructions for a particular protein or enzyme is “transcribed” into a corresponding sequence of RNA. The RNA sequence in turn is “translated” into the sequence of amino acids which form the protein. Each amino acid is represented in DNA or RNA by one or more triplets of nucleotides, called a codon. The genetic code has some redundancy, also called degeneracy, meaning that most amino acids have more than one corresponding codon corresponding to an amino acid. The amino acid lysine (Lys), for example, can be coded by the nucleotide triplet or codon AAA or by the codon AAG. Codons may also form translation stop signals (e.g., TGA, TAG or TAA). Because the nucleotides in DNA and RNA sequences are read in groups of three for protein production, it is important to begin reading the sequence at the correct nucleotide, so that the correct triplets are read. The way that a nucleotide sequence is grouped into codons is called the “reading frame.”

The term “gene” refers to a DNA sequence that encodes or corresponds to a particular sequence of amino acids that comprise all or part of one or more proteins, and may or may not include regulatory DNA sequences, such as, for example, promoter sequences, which determine, for example, the conditions under which the gene is expressed. The term “gene” also includes DNA sequences which are transcribed from DNA to RNA, but are not translated into an amino acid sequence.

A “coding sequence” or a sequence “encoding” an expression product, such as a RNA, polypeptide, or protein, is a nucleotide sequence that, when expressed, results in the production of that RNA, polypeptide, or protein, i.e., the nucleotide sequence encodes an amino acid sequence for that polypeptide or protein. A coding sequence for a protein may include a start codon (usually ATG) and a stop codon. A nucleic acid may also “encode” a gene or DNA sequence in that the nucleotide sequence of the gene or DNA sequence is contained within the nucleic acid.

A “promoter sequence” is a DNA regulatory region capable of binding RNA polymerase in a cell or in vitro and initiating transcription of a downstream (3′ direction) coding sequence. A promoter sequence is bounded typically at its 3′ terminus by the transcription initiation site and extends upstream (5′ direction) to include bases or elements necessary to initiate transcription at higher or lower levels than that of a promoter without said bases or elements. Within the promoter sequence may be found a transcription initiation site (conveniently defined, for example, by mapping with nuclease S1), as well as protein binding domains (consensus sequences) responsible for the binding of RNA polymerase.

A coding sequence may be “under the control of”, “operatively associated with” or “functionally associated with” transcriptional and translational control sequences in a cell when RNA polymerase transcribes the coding sequence into mRNA, which may then be spliced (if it contains introns) and may also be translated into the protein encoded by the coding sequence.

The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a protein. The expression product itself, e.g. the resulting protein, may also be said to be “expressed” by the cell. An expression product can be characterized as intracellular, extracellular or secreted. The term “intracellular” means something that is inside a cell. The term “extracellular” means something that is outside a cell. A substance is “secreted” by a cell if it appears in significant measure outside the cell, from somewhere on or inside the cell.

The term “gene transfer” refers broadly to any process by which nucleic acids are introduced into a cell. Accordingly, the term “gene therapy” refers to the use of a gene transfer process, preferably, for the purpose of causing a therapeutic effect in a patient. Transfer of an HAS gene (e.g., HAS1, 2 or 3) into the cells of a subject (e.g., ocular cells) constitutes a gene transfer. Transfer of a HAS gene into the ocular cells of a patient for the purpose of treating DES is a gene therapy process.

The term “transfection” or “transformation” means the introduction of a foreign nucleic acid into a host cell by any means. Transfection or transformation may cause the host cell to express a gene or sequence which has been introduced to produce a desired substance, typically a protein coded by the introduced gene or sequence. The introduced gene or sequence may also be called a “cloned” or “foreign” gene or sequence and may include regulatory or control sequences, such as start, stop, promoter, signal, secretion, or other sequences used by a cell's genetic machinery. The gene or sequence may include nonfunctional sequences or sequences with no known function. The DNA or RNA introduced to a host cell can come from any source, including cells of the same genus or species as the host cell, or cells of a different genus or species.

The term “vector” means the vehicle by which a DNA or RNA sequence (e.g., a foreign gene) can be introduced into a host cell, so as to transform or transfect the host. Transformation or transfection may promote expression (e.g., transcription and translation) of the introduced sequence. Vectors may include plasmids (e.g., pcDNA 3.1/HisC12-13).

Vectors typically comprise the DNA of a transmissible agent, into which foreign DNA is inserted. A common way to insert one segment of DNA into another segment of DNA involves the use of enzymes called restriction enzymes, which cleave DNA at specific sites (specific groups of nucleotides) called restriction sites, and DNA ligase which joins pieces of DNA, such as a restriction enzyme digested nucleic acid and a restriction enzyme digested plasmid vector, together. A “cassette” refers to a DNA coding sequence or segment of DNA that codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.” A common type of vector is a “plasmid”, which generally is a self-contained molecule of double-stranded DNA, usually of bacterial origin, that can readily accept additional (foreign) DNA and which can be readily introduced into a suitable host cell. A plasmid vector often contains coding DNA and promoter DNA and has one or more restriction sites suitable for inserting foreign DNA as well as an origin of replication. Promoter DNA and coding DNA may be from the same gene or from different genes, and may be from the same or different organisms. A large number of vectors, including plasmid and fungal vectors, have been described for replication and/or expression in a variety of eukaryotic and prokaryotic hosts. Non-limiting examples include pcDNA3.1, pcDNA/HisC 12-13, pKK plasmids (Clonetech), pUC plasmids, pET plasmids (Novagen, Inc., Madison, Wis.), pRSET or pREP plasmids (Invitrogen, San Diego, Calif.), or pMAL plasmids (New England Biolabs, Beverly, Mass.), and many appropriate host cells, using methods disclosed or cited herein or otherwise known to those skilled in the relevant art. Recombinant cloning vectors will often include one or more replication systems for cloning or expression, one or more markers for selection in the host, e.g. antibiotic resistance, and one or more expression cassettes.

The term “sequence identity” or “identity” refers to exact matches between the nucleotides or amino acids of a two nucleic acids or proteins, respectively, when these sequences are compared. For example, the degree of nucleotide sequence identity between two nucleic acids may be determined by comparison of the sequences with a BLASTN or CLUSTALW sequence comparison algorithm. Similarly, identity between the amino acid sequences of two proteins may be determined by use of a BLASTP or CLUSTALW sequence comparison algorithm. The BLAST algorithms are commonly known in the art and are publically accessible, at no cost, at the National Center for Biotechnology Information website (www.ncbi.nlm.nih.gov). The CLUSTALW algorithm is publically accessible, at no cost, at the European Bioinformatics Institute website (www2.ebi.ac.uk/clustalw/).

As used herein, the term “sequence similarity”, “similarity”, “sequence homology” or “homology” refers to both the exact matches and conserved matches between the amino acid sequences of two proteins. Sequence homology between the amino acid sequences of two proteins may be determined using a CLUSTALW or BLASTP algorithm. A conserved match is a match between two amino acids which are of similar biochemical classification and/or biochemical properties. For example, in the context of a protein sequence comparison, a match of one amino acid with a hydrophobic side group with a different amino acid with a hydophobic side group would be considered a conserved match. Non-limiting examples of biochemical classes which are generally known by those skilled in the art are as follows: hydrophobic (valine, leucine, isoleucine, methionine, phenylalanine, tryptophan, alanine, proline); hydrophilic (histidine, lysine, arginine, glutamic acid, aspartic acid, cysteine, asparagine, glutamine, threonine, tyrosine, serine, glycine); no charge/hydrophilic (cysteine, asparagine, glutamine, threonine, tyrosine, serine, glycine); aromatic (tryptophan, tyrosine, phenylalanine); negatively charged/hydrophilic (aspartic acid, glutamic acid); positively charged/hydrophilic (histidine, lysine, arginine).

The BLAST algorithms are commonly known in the art. The following references regarding the algorithms are herein incorporated by reference: BLAST ALGORITHMS: Altschul, S. F., et al., J. Mol. Biol. 215:403-410, 1990; Gish, W., et al., Nature Genet. 3:266-272, 1993; Madden, T. L., et al., Meth. Enzymol. 266:131-141, 1996; Altschul, S. F., et al., Nucleic Acids Res.25:3389-3402,1997; Zhang, J., et al., Genome Res. 7:649-656,1997; Wootton, J. C., et al, Comput. Chem. 17:149-163, 1993; Hancock, J. M. et al., Comput. Appl. Biosci. 10:67-70, 1994; ALIGNMENT SCORING SYSTEMS: Dayhoff, M. O., et al.,“A model of evolutionary change in proteins.” In Atlas of Protein Sequence and Structure, vol. 5, suppl. 3. M. O. Dayhoff (ed.), pp. 345-352, Natl. Biomed. Res. Found., Washington, D.C., 1978; Schwartz, R. M., et al., “Matrices for detecting distant relationships.” In Atlas of Protein Sequence and Structure, vol. 5, suppl. 3. M. O. Dayhoff(ed.), pp.353-358, Natl. Biomed. Res. Found., Washington, D.C., 1978; Altschul, S. F., J. Mol. Biol. 219:555-565, 1991. States, D. J., et al., Methods 3:66-70, 1991. Henikoff, S., et al., Proc. Natl. Acad. Sci. USA 89:10915-10919, 1992; Altschul, S. F., J. Mol. Evol. 36:290-300,1993; ALIGNMENT STATISTICS: Karlin, S., et al., Proc. Natl. Acad. Sci. USA 87:2264-2268, 1990; Karlin, S., et al., Proc. Natl. Acad. Sci. USA 90:5873-5877, 1993; Dembo, A., et al., Ann. Prob. 22:2022-2039, 1994; and Altschul, S. F., “Evaluating the statistical significance of multiple distinct local alignments.” In Theoretical and Computational Methods in Genome Research.” (S. Suhai, ed.), pp. 1-14, Plenum, N.Y., 1997.

The present invention includes bioconjugates which comprise nucleic acids comprising a hyaluronan synthase gene (i.e., a gene comprising hyaluronan synthase activity), preferably the nucleic acid comprises a nucleotide sequence of at least about 70% identity to a reference nucleotide sequence selected from the group consisting of SEQ ID NOs. 1-3 (preferably at least about 85% sequence identity to SEQ ID NO. 1, at least about 81% sequence identity to SEQ ID NO. 2 and at least about 79% sequence identity to SEQ ID NO. 3) as well as nucleic acids which have a nucleotide sequence which encodes a hyaluronan synthase enzyme, preferably, wherein the encoded amino acid sequence comprises at least about 70% homology or identity to a sequence selected from the group consisting of SEQ ID NOs. 4-6, (preferably at least about 66% sequence homology to SEQ ID NO. 4, at least about 90% sequence homology to SEQ ID NO. 5 and at least about 89% sequence homology to SEQ ID NO.6.) wherein identity or homology is determined using the BLASTN or BLASTP algorithms, respectively, where the parameters of the algorithms are selected to give the largest match between the respective sequences tested, over the entire length of the respective reference sequences. However, in preferred embodiments, the level of identity or homology mentioned above is greater than about 70%, preferably about 80% or greater, more preferably about 90% or greater, even more preferably about 95% to about 99% or greater and most preferably about 100%.

The present invention also includes bioconjugates which comprise nucleic acids comprising a hyaluronan synthase gene comprising a nucleotide sequence of at least about 70% identity to a reference nucleotide sequence of any hyaluronan synthase gene disclosed in U.S. patent application No. 08/812,008 and U.S. patent application No. 08/675,499, which are herein incorporated by reference in their entireties, as well as bioconjugates comprising nucleic acids which encode hyaluronan synthase proteins comprising amino acid sequences with at least about 70% homology or identity to the hyaluronan synthase protein amino acid sequences disclosed in the incorporated applications wherein identity or homology is determined using the BLASTN or BLASTP algorithms, respectively, where the parameters of the algorithms are selected to give the largest match between the respective sequences tested, over the entire length of the respective reference sequences. However, in preferred embodiments, the level of identity or homology mentioned above is greater than about 70%, preferably about 80% or greater, more preferably about 90% or greater, even more preferably about 95% to about 99% or greater and most preferably about 100%.

The term “induce” or “induction” refers to an increase by a measurable amount.

### Derivatized Hyaluronic Acid/Nucleic Acid Bioconjugate

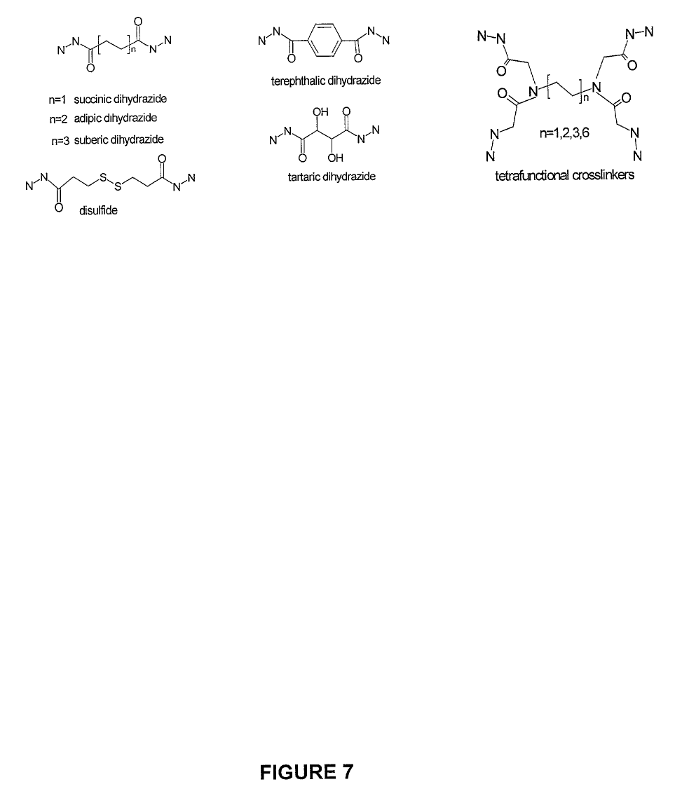

The dihydrazide derivatized hyaluronic acid/nucleic acid bioconjugates of the invention include hyaluronic acid which has been derivatized with a dihydrazide; a nucleic acid may be crosslinked to this derivative at any locus on the dihydrazide derivatized hyaluronic acid molecule. A dihydrazide modified hyaluronic acid in which a pendent hydrazide moiety is still available for reaction may be referred to as a hydrazido hyaluronic acid. Alternatively, a derivatized hyaluronic acid/nucleic acid which has been conjugated to a nucleic acid by way of any chemical crosslinker (e.g., carbodiimides) is within the scope of the present invention. Without being bound by a single theory, it is believed that the dihydrazide derivatized hyaluronic acid/nucleic acid bioconjugate of the invention may include hyaluronic acid which is crosslinked to a dihydrazide wherein the a dihydrazide portion of the molecule is further crosslinked to a nucleic acid. Adipic dihydrazide is the preferred dihydrazide with which to derivatize hyaluronic acid, however, other dihydrazide molecules may be used for this purpose (see FIG. 7). Said nucleic acids may be in the form of linear DNA, oligonucleotides or RNA, however, in a preferred embodiment, the nucleic acid is plasmid DNA. Accordingly, a preferred embodiment of the invention includes bioconjugates comprising plasmid DNA conjugated to adipic dihydrazide derivatized hyaluronic acid. The bioconjugates of the invention may be further conjugated to other substances such as other small molecules, antibodies, proteins and peptides. These additional substance may impart an additional therapeutic functionality upon the bioconjugates of the invention. The bioconjugates of the invention may be further conjugated to ligands which allow the bioconjugates to be targeted to a particular location in the subject's eye. For example, this location may comprise a particular type of cell such as conjunctival cells in the conjunctival tissue. Furthermore, the additional conjugates may be used to prevent or inhibit the bioconjugates of the invention from contacting certain cell types or tissues.

The term “derivative” and all of its grammatical forms refers to a compound obtained from a parent substance which contains most or all of the essential elements of said parent substance.

“Dihydrazide” refers to molecules having the formula:

H₂N—NH—C(═O)—R—C(═O)—NH—NH₂;

wherein R is any compound. Carbodihydrazides, wherein R is an aliphatic group (e.g., methyl-, ethyl-, propyl-, isopropyl-, butyl-, pentyl-, hexyl-, heptyl-, or octyl) are preferred, however other dihydrazides are within the scope of the invention, such as sulfonodihydrazides and phosphonicdihydrazides. Adipic dihydrazide refers to H₂N—NH—C(═O)—(CH₂)₄—C(═O)—NH—NH₂; chemical abstracts registration number 1071-93-8. FIG. 7 illustrates non-limiting examples of preferred dihydrazides that may be used in the present invention.

The term “carbodiimide” refers to compounds with the general formula R—N═C═N—R′ wherein R and R′ may be any moiety, preferably a hydrocarbon such as a methyl-, ethyl-, propyl-, ispropyl-,butyl-, pentyl-, hexyl- or cyclohexy- group. In preferred embodiments, the carbodiimide used in the present invention comprise EDAC (1-ethyl-3-(3-dimethyl-aminopropyl) carbodiimide, BCC (N,N′-dicyclohexyl carbodiimide), DIC (diisopropylcarbodiimide) or EDC.

The term “moiety” refers to a chemical entity which may be a part, portion or subunit of a larger entity.

The term “crosslinked” or “conjugated” refers to the attachment of two substances via any type of bond or force. A non-limiting list of specific means by which to crosslink two substances may include covalent bonds, ionic bonds or hydrogen bonds, van der Waals forces, ionic interactions and hydrophobic interactions.

The dihydrazide derivatized hyaluronic acid/nucleic acid bioconjugate of the invention may be formulated to a variety of dosage forms. Nucleic acids can be released from cross-linked HA microspheres, films, wafers, matrices, hydrogels, gels, sols, or any other form of the bioconjugates of the invention. All of these may find application in ophthalmic practice. The term “gel” is intended to refer to viscous or semi-solid and jelly-like material. The term “hydrogel” is intended to mean macromolecular networks, which swell in water. The term “film” is intended to mean a substance formed by compressing a gel or by allowing or causing a gel to dehydrate. The term “matrix” is intended to mean a substance formed by lyophilizing a gel. The term “microsphere” refers to microscopic particles used to deliver substances, such as nucleic acids, to a target cell. The microspheres of this invention may have a diameter (e.g., less than about a millimeter). In preferred embodiments, the microspheres of the invention are between about 1 μm and about 100 μm, more preferably between about 15 μm and about 25 μm. However, microspheres of any size wherein the essential elements of the present invention are preserved are within the scope of this invention. A wafer is a matrix like structure which generally has a weight ranging from about 1 to about 100 mg and has a defined shape such as a circle or square. The derivatized hyaluronic acid/nucleic acid bioconjugates may include any gene. Preferred embodiments include a gene which produces hyaluronic acid or produces a substance which causes the production of hyaluronic acid; in preferred embodiments, this gene is hyaluronan synthase (e.g., HAS 1, HAS 2 or HAS 3). Derivatized hyaluronic acid/nucleic acid bioconjugates that include said genes may also include, within the nucleic acid that contains the gene, additional nucleotides whose sequence causes expression of a protein or RNA, which corresponds to the gene, in a cell.

Accordingly, bioconjugates which include derivatized hyaluronic acid that is conjugated to plasmid DNA that encodes the hyaluronan synthase gene is a preferred embodiment of the invention.

The preparation of nucleic acids which may be used in the present invention may be accomplished by any means which yields nucleic acids of sufficient quality and purity so as to allow the successful practice of the invention. For example, nucleic acids may be produced by cesium chloride purification, chromatographic purification (e.g., with anion exchange resins), PEG precipitation or by simple alkali lysis/ethanol precipitation purifications. These methods are all commonly known in the art. Several commercially available purification kits are available which may used to prepare nucleic acids for use in the present invention (e.g., Qiagen plasmid prep kits (Qiagen, Inc.; Valencia, Calif.)).

The covalently crosslinked derivatives of hyaluronic acid yield hydrogels with enhanced rheological and mechanical properties with differences in hyaluronidase mediated degradation and thus differences in the kinetics of DNA release. The conjugate of hyaluronic acid and nucleic acid gels or hydrogels may be topically applied for sustained gene transfer.

### Formulations and Administration

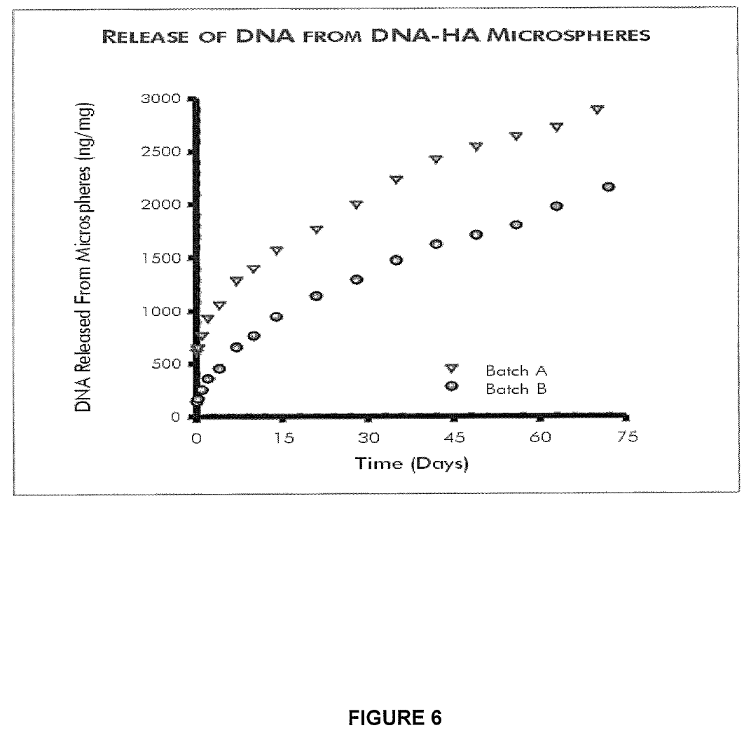

An important goal of formulation science is the maintenance of potency and stability over time during storage under conditions usually encountered by drugs and biologicals. Lyophilization and vitrification are well known methods of achieving this in the pharmaceutical industry. Further, prior to lyophilization or vitrification, formulations may be produced containing substances known to confer stability to biologicals and drugs. These formulations usually contain a physiological buffer and/or salts such as sodium chloride, sugars such as maltose, dextrose or xylose, or polymers such as dextrans or celluloses. To make the dosage form, several may be used. A volume of the HA-DNA solution containing the appropriate gene dosage could be placed into a series of molds. The measured solution would be frozen to appropriate temperatures for the lyophilization step, then lyophilized. After lyophilization, the required cross-linking solution containing the cross-linker, carbodiimide, organic solvent/water solution could be introduced directly into the mold containing the dosage unit. After crosslinking, subsequent processing, as described in FIG. 6, could be performed. After the last step, the dosage unit could be extracted and packaged using conventional blister packaging methods for example, well known to the medical device and pharmaceutical industry. The packaged dosage forms may be sterilized by an appropriate method (e.g., ETO, EB, gamma irradiation). Alternatively, the dosage form may be manufactured by an aseptic batch process using sterile (filter sterilized) solutions of reagents and hyaluronan. The sterile solution is dispensed into the single-dose mold, then lyophilized and processed as described above. Aseptic lyophilization processes are routinely practiced by the pharmaceutical and medical device industries.

The sterile, packaged dosage form is stored at room temperature. At the point of use, the package is opened and the dosage form is rehydrated with an appropriate ophthalmic solution. It is then placed in or on the eye in an appropriate position that will allow gene transfer to occur.

Specifically, solid dosage forms such as films, wafers and matrices may be handled with appropriate instruments for placement in the eye much like contact lenses. These materials are typically rehydrated with a suitable solvent such as buffers or solutions routinely used in ophthalmic practice prior to implantation (e.g., distilled water, phosphate buffer and/or saline). Sols or gels may be applied using syringes, droppers or other devices capable of delivering a metered amount of liquid material. Microspheres, if dried or lyophilized, may be resuspended in solvents (e.g., distilled water phosphate buffer and/or saline) used in ophthalmic practice and applied as described for gels and sols.

The biconjugate may be placed between the eye and conjunctiva for a period of time which is sufficient to allow a gene transfer process to occur (e.g., 1 hour, 5 hours or 10 hours). Alternatively, the bioconjugate may be placed directly over the surface of the eye, after which the eye lid may be closed and covered with a patch. In this embodiment, the bioconjugates may be allowed to incubate for an extended period (e.g., 12 or 24 hours), after which the patch may be removed and the eye lid may be opened.

### Effective Dose

The present invention comprises embodiments wherein any dose of the bioconjugates of the invention is given to a patient. Preferably, the dose given to the patient is sufficient to cause a therapeutic effect. In severe cases, patients may require a large amount of new hyaluronan to be produced. In these cases, a physician may apply two doses simultaneously to increase the number of eye cells transfected, or administer the standard dose more frequently.

### Therapeutic Uses of Derivatized Hyaluronic Acid/Nucleic Acid Bioconjugates

The derivatized hyaluronic acid/nucleic acid bioconjugates of the invention may be administered to a patient for the purpose of treating dry eye syndrome. In the treatment of dry eye syndrome, some of the eye cells of the patient are transfected with a hyaluronan synthase gene (e.g., HAS1, 2 or 3) by contacting the target cells with the derivatized hyaluronic acid/nucleic acid bioconjugates of the invention. The term “dry eye syndrome” (or “DES”) refers to any medical condition caused by a lack of moisture in the eye. The hyaluronan synthase genes are biologically active since they are coupled to axillary genetic elements which allow the expression of the hyaluronan synthase gene to occur in the target cells. The genetic construct used in the treatment of DES may comprise an expression plasmid (a closed, circular piece of DNA) having a suitable promoter which allows the plasmid to direct the synthesis of the HAS protein. The promoter may be a human tissue specific promoter which will function in the target cell. An example of a promoter which may be used in this invention is an epithelial cell promoter (e.g., the ED-LIE promoter), a human papilloma virus promoter, a B2LF1 promoter from Epstein Barr Virus or a Cytomegalovirus (CMV) promoter. The construct may also have enhancer elements which increase the efficiency of transcription and translation and other elements which help stabilize the structure and function of the element in the cell. Without being bound by theory, it is believed that natural hyalurondases associated with the eye, as well as hydrolysis, degrades the hyaluronic acid portion of the bioconjugates and liberates the nucleic acid. The free nucleic acid is then available for uptake by the target eye cells.

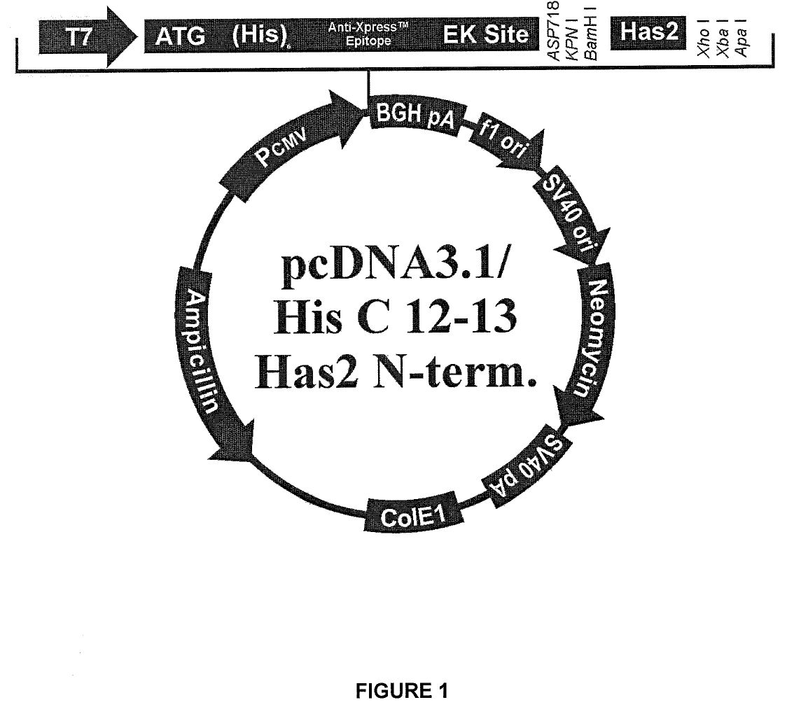

An example of an experimental construct which may be used to express hyaluronan synthase in cells is shown in FIG. 1. Expression of the gene in the ocular tissues will increase the hyaluronic acid content of the tear film of the eye. This will ease the symptoms of dry eye. Another aspect relates to the provision of genes to other tissues of the eye that would benefit from localized therapy. Diseases of the retina, for example, could be treated by application of a matrix or wafer containing genes or antisense oligonucleotides that would inhibit angiogenesis for the treatment of macular degeneration or genes related to lipid biosynthesis that would help to restore the lipid component of the tear film. Specifically, the conjugate of the present invention may be used to treat medical conditions of the eye wherein delivery of a nucleic acid to the cells of the eye would have a desirable therapeutic effect. For the purposes of the present application, the term “eye” refers to the visual organ as commonly known including all tissues and substructures thereof. For example, “eye” includes, but is not limited to, epithelial cells, optic nerve, retina, lens, cornea, iris, sclera, choroid, cilliary body, conjunctiva, vitreous humor and aqueous humor. In a preferred embodiment, the cells of the conjunctiva are transfected by the conjugates of the invention.

### In Vitro Transformation of Cells

Transformation. The bioconjugates of the present invention are useful reagents for in vitro transformation of any cell, preferably a eukaryotic cell, more preferably a human eye cell (e.g, a conjunctival or corneal epithelial cell) with a nucleic acid which, preferably, comprises a HAS gene (e.g. HAS1-3). Preferably the nucleic acid has a nucleotide sequence of about 70% to about 100% identity to a reference nucleotide sequence selected from the group consisting of SEQ ID NOs. 1-3 or wherein the nucleic acid has a nucleotide sequence which encodes a hyaluronan synthase enzyme (e.g, HAS1-3), preferably the amino acid sequence of the enzyme comprises about 70% to about 100% homology or identity to a reference amino acid sequence selected from the group consisting of SEQ ID NOs.4-6 wherein homology is determined using the BLASTN or BLASTP algorithm, respectively, where the parameters of the algorithm are selected to give the largest match between the sequences tested over the entire length of the reference sequence

The bioconjugates provide a means by which a nucleic acid may be gradually introduced, over a long period of time, to cells in culture. As the hyaluronic acid portion of the bioconjugate is degraded, the nucleic acid portion of the bioconjugate is liberated and made available for uptake by the host cells.

When the bioconjugates are used for in vitro cell transformation, the host cells are grown in a manner similar to that of conventional transformation protocols which are commonly known in the art. The protocols are altered, however, to include addition of the bioconjugates instead of, or in addition to, free, unconjugated, nucleic acids. For example, liposome mediated transformation protocols using the bioconjugates may include seeding a 35 mm plate with about 2×10⁵ cells and growth of the cells to about 40%-80% confluency (e.g, 40%, 50%, 60%, 70% or 80%). After growth, a mixture comprising the bioconjugates (e.g, comprising 1 μg or more of nucleic acid), hyaluronidase (e.g, 10, 15, 20, or 25 units/ml bovine testicular hyaluronidase) and cationic liposomes (e.g., lipofectin) are added to the cells and the cells are incubated for about 24 to about 72 hours (e.g,. 30 hours, 40 hours, 50 hours, 60 hours or 70 hours). The hyaluronidase is believed to degrade the hylauronic acid of the bioconjugate and release the nucleic acid; the rate at which the nucleic acid is released may be modulated by controlling the amount of hylauronidase which is used. After incubation, the nucleic acids which were conjugated in the bioconjugates have entered and transformed the host cells.

Hyaluronic acid determination. One way to determine the level of hyaluronan synthase activity in a cell is to determine the level of hyaluronic acid in the cell. Cells with a high level of hyaluronan synthase activity may comprise a high level of hyaluronic acid. Furthermore, the level of hyaluronan synthase activity which is associated with a given gene or protein (e.g., human, Xlaevis, B. taurus, M.musculus, R. norvegicus or G.gallus, HAS1, HAS2 or HAS3) can be determined by introducing the gene (e.g., by use of the bioconjugates of the invention) into a cell (e.g., on ocular cell) and then determining the level of hyaluronic acid in the cell. The level of hyaluronic acid in the cell may be compared to that of a cell into which a gene has not been introduced. Determination of hyaluronic acid or hyaluronate in a sample is a practice which is commonly known in the art. Hyaluronic acid production, in cells which have been transformed with the bioconjugates of the invention, may be determined by measuring the level of hyaluronic acid in the cells by any conventional method known in the art.

For example, hyaluronic acid in a transformed cell may be measured by a Particle Exclusion Assay. The Assay includes contacting transformed cells with fixed and suspended erythrocytes; the erythrocytes maybe obtained commercially. After the cells are allowed to settle they may be examined microscopically. Transformed cells which express hyaluronic acid on the cell surface will form a zone which excludes the erythrocytes. The exclusion zone will be visible under the microscope and will indicate that the cells are producing hyaluronic acid on their surface.

Hyaluronic acid production by transformed cells may also be measured by a Biotinylation Reporter Assay. In this assay, transformed cells are contacted with biotinylated Hyaluronic Acid Binding Protein (biotin-HABP), which is commercially available from Segugaku (Japan), and then unbound biotin-HABP is washed away. Biotin-HABP will bind to the surface of the cells if hyaluronic acid is present. After binding, streptavidin conjugated alkaline phosphatase in added to the biotin-HABP bound cells and an alkaline phosphatase substrate is added. The streptavidin moiety of streptavidin conjugated alkaline phosphatase will bind to the biotin moiety of biotin-HABP on the surface of the cells. The presence of the biotin-HABP-streptavidin-alkaline phosphatase complex on the cells will be apparent when alkaline phosphatase catalyzes the substrate to produce a colored product. Preferably, the substrate forms an insoluble product, when catalyzed by alkaline phosphatase, which deposits on the surface of the cell. The color change may be observed microscopically or it can be measured colorimetrically in a clear plastic microtiter plate using a microplate reader.

Hyaluronan synthase activity may also be determined using the assays disclosed in U.S. Pat. No. 5,378,637; U.S. Pat. No. 4,826,776; U.S. Pat. No.5,019,498; Larjava H, et al., Arch. Dermatol. Res. 273(3-4):199-2,1982; DeAngelis, P. L. et al.,Biochemistry 33: 9033-9039, 1994 or Pummill P. E., et al., J. Biol. Chem. 273:4976-4981, 1998 which are herein incorporated by reference in their entireties.

### Manufacture of Derivatized Hyaluronic Acid/Nucleic Acid Bioconjugates

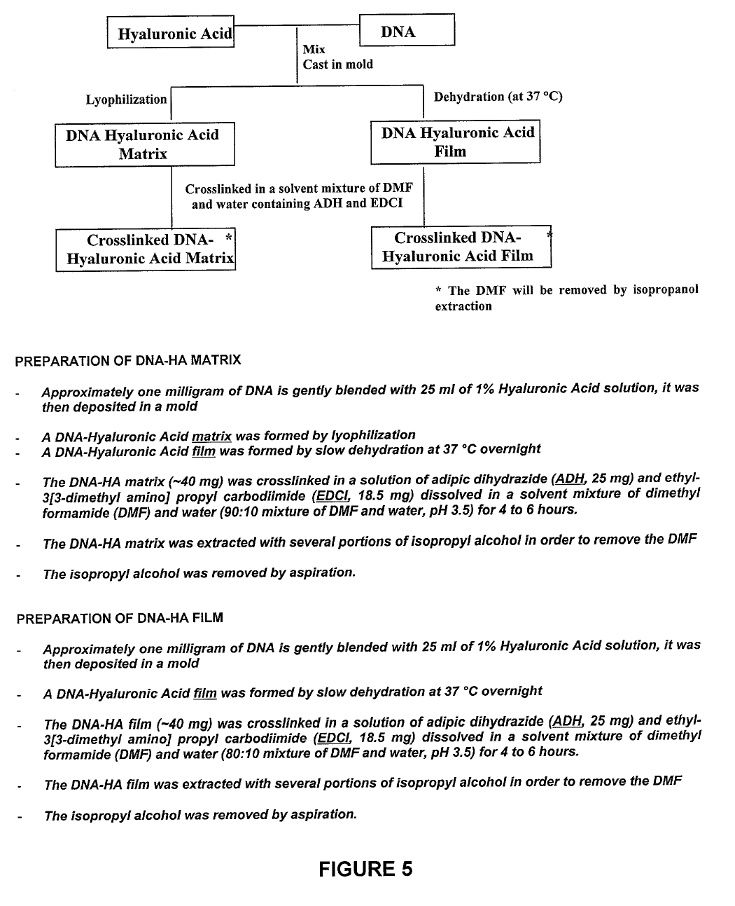

Another aspect relates to the commercial feasibility of manufacture of the bioconjugates. A prototype manufacturing protocol has been established. Essentially, devices can be made as follows: An aqueous solution of HA is mixed with the DNA, preferably a plasmid, to be delivered. The solution is then frozen and lyophilized. The lyophilized material is then immersed in a solution of 90% dimethyl formamide or similar solution of an alcohol containing a dihydrazide crosslinker, and a carbodiimide. The pH of the solution is adjusted to an acidic range below 7, and preferably between about 1 to about 5, and the HA-DNA mixture is allowed to become crosslinked. After a predetermined amount of time, the lyophilized material is removed and immersed in aqueous alcohol to remove unreacted components and to sterilize the material. This is a batch process that is very scalable and the degree of crosslinking can be controlled by pH, and reactant concentration. FIG. 5 illustrates a flow chart depicting the method described above.

Gels and sols having approximately 97-99.5% (w/v) water content are made by mixing a solution of hyaluronan and DNA with a suitable carbodiimide and cross-linker, then reducing the pH of the solution with an acid to a range that will not solidify the mixture but would render it highly viscous or gel-like. This range is typically pH 4-4.75. A firmer, shape holding gel can be made by further lowering the pH of the mixture to 2.0. Films can be made by allowing cross-linked gels (as described above) to dehydrate and lose moisture at atmospheric pressure or reduced pressure. Matrices and wafers are made by either mixing all of the components together, cross-linking them by lowering the pH of the mixture and lyophilizing the resultant formulation, or by mixing the HA and DNA together, lyophilizing the mixture, then subsequently cross-linking the resultant “matrix” or “wafer” as described above in an alcoholic or DMF solution of cross-linker and carbodiimide. This process is illustrated in FIG. 6.

One aspect of the invention relates to the control of the release of DNA by the extent of cross-linking to which DNA-HA complexes are subjected. Extensive cross-linking delays the release of DNA over time and creates a sustained release dosage form, while superficial or “mild” cross-linking allows the DNA to be released rapidly. The extent of cross-linking can be controlled by the concentration of the reactants, the pH of the reaction mix, the molecular weight of the hyaluronan (Vercruysse, K. P., et al., Bioconjugate Chemistry, 8(5):686-694, 1997) and the amount of time the hyaluronic acid is allowed to crosslink in the presence of crosslinker.

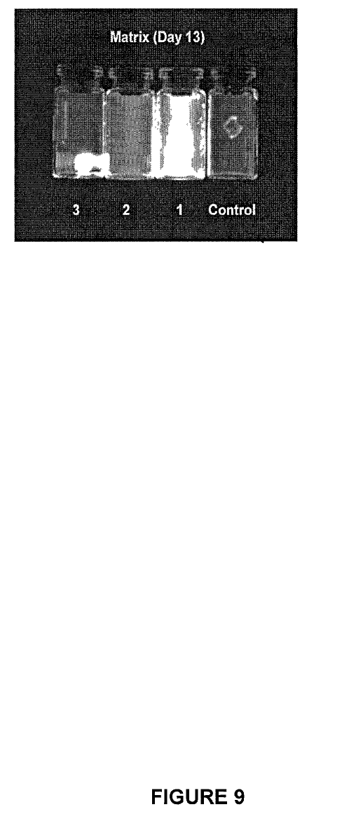

The extent of crosslinking may be increased by decreasing the pH of the crosslinking reaction, extending the period of time for which the hyaluronic acid is allowed to crosslink in the presence of crosslinker or by increasing the concentration of the crosslinking reagent (e.g., the dihydrazide) in the reaction. Conversely, the extent of crosslinking may be decreased by increasing the pH of the crosslinking reaction, shortening the period of time for which the hyaluronic acid is allowed to crosslink in the presence of crosslinker or by decreasing the concentration of the crosslinking reagent in the crosslinking reaction. FIG. 6 demonstrates that DNA release from microspheres including HA which was incubated in the presence of crosslinker for 16 hours (batch B) is more prolonged than for that of microspheres including HA incubated in the presence of crosslinker for only 6 hours (batch A). Similarly, FIG. 9 demonstrates that DNA/HA matrices including HA incubated in the presence of crosslinker for 6 hours (#2) are less refractory to degradation and release of DNA than that of matrices including HA crosslinked for 12 hours (#1) or 24 hours (#3).

The release of DNA can also be controlled by using a variety of cross-linkers which confer different degrees of resistance to hydrolysis by hyaluronidases. Terephtalate dihydrazide produces gels which are very resistant to degradation by hyaluronidase while adipic dihydrazide produces gels that are more easily degraded. The kinetics of DNA release can be evaluated with an assay, commonly known in the art, that measures the amount of DNA released over time in the presence of a fixed amount of hyaluronidase enzyme. The temperature is generally held constant throughout the assay. Using the assay, the effects of different cross-linkers, reactant concentrations, DNA loadings and cross-linking conditions on the release of DNA can be evaluated. The assay described in this invention can also be performed in the absence of hyaluronidase to determine the kinetics of the non-enzymatic release of DNA. An example of the results that can be obtained with the assay is shown in FIG. 6.

For the purposes of this application the process of drying materials refers to any method which causes the level of moisture in a suspension or solution, whether the suspension or solution is aqueous or non-aqueous, to decrease. In preferred embodiments, the method of drying is lyophilization. Lyophilization refers to the process of freezing a liquid and drying it under a vacuum.

### Examples

The invention may be better understood by reference to the following examples, which is provided by way of exemplification and not limitation.

### Example 1

Expression of Murine Hyaluronan Synthase Genes in Mammalian Cells

Plasmid DNA constructs capable of expressing the mouse hyaluronan synthase (HAS) genes HAS2 and HAS3 in mammalian cells were prepared as follows. Plasmids encoding the mouse hyaluronan synthase cDNAs HAS2 (SEQ ID NOs: 2 and 5; Spicer et al., J. Biol. Chem., 271:23400-23462, 1996) and HAS3 (SEQ ID NOs: 3 and 6; Spicer et al., J. Biol. Chem., 273:1923-1932, 1998) were digested with restriction endonucleases SmaI, and EcoRI, respectively, to release their inserts. The fragments were separated by electrophoresis on agarose gels, and the bands containing the insert excised. The insert DNA was purified from the gel slices by solubilizing in a chaotropic buffer, binding to a commercially available (Qiagen; Valencia, Calif.) silica matrix spin filter, followed by elution with a Tris buffer at pH8.5.

The isolated inserts were then used as templates for a PCR reaction. PCR forward primers were designed to conserve the previously inserted optimized Kozak initiation sequence (Kozak M, Nucl. Acids Res., 12:857-872, 1984), as well as add a new restriction enzyme site (BamHI) for cloning. Reverse primers were also designed to incorporate a new restriction enzyme site (XhoI). In addition, two versions of the reverse primers were made; one containing a stop codon at the termination of the HAS cDNA sequence, and another minus the stop codon allowing in-frame read through to a C-terminal epitope tag (V5-His). A high fidelity thermostable polymerase (pfu Turbo, Stratagene; La Jolla, Calif.) was used to minimize the error rate. The resulting PCR products were analyzed by gel electrophoresis and purified (Qiagen; Valencia, Calif.). All four sets of PCR products were digested sequentially with XhoI and BamHI, and purified.

A mammalian expression vector pcDNA3.1/V5-His A (Invitrogen; Carlsbad, Calif.) that utilizes the CMV promoter to drive expression was chosen. The vector was double cut with XhoI and BamHI, and gel purified as above for the inserts. Purified digested vector was annealed and ligated to the digested PCR products. Ligation mixes were used to transform competent E. coli bacteria followed by plating on carbenicillin LB agar. Transformants were picked, cultured in selective media and plasmid DNA was isolated. The plasmids were examined for the presence of the PCR product sequence by restriction analysis and DNA sequencing. Expression of the constructs with the C-terminal V5-His epitope tag was also tested by transfection into CHO-K1 cells. Immunohistochemical staining with an anti-V5 antibody (V5 is a viral epitope engineered into the expressed protein, and is not an epitope known to exist in mammalian cells) detected recombinant HAS2 and HAS 3 and demonstrated that the recombinant proteins were expressed in the host cells.

### Example 2

Formulation of the Bioconjugates of the Invention

Medical grade hyaluronan powder was obtained from several suppliers (Kraeber; Germany, Genzyme; Cambridge, Mass.) and was hydrated in sterile water for 16 hours with gentle stirring using a variable speed mixer and flat blade propeller. A sterile reaction vessel with a cover and with ports that allow the introduction of reagents aseptically and a sterile propeller was used. The volume of the solution was about 90% of the final volume to allow for the addition of other reagents. The final weight of HA per volume was 1%. After cooling to ambient temperature, the DNA expression plasmid of EXAMPLE 1 was added and mixed well at slow speed (about 100 rpm). To effect a 1% weight per volume loading, 10 mg of DNA was added per gram of HA. If no buffers or other reagents are required and matrices are to be made (see FIG. 6), the liquid mixture may be dispensed into a mold that will contain one dose or multiple doses of the therapeutic. Alternatively, the mixture may be placed into a mold that will form the product for subsequent division into the desired dosage form. After dispensing, the filled mold was frozen to a temperature suitable for lyophilization (approximately −50° C.). The lyophilization process was then carried out for 36 hours. Afterwards, the product was crosslinked by the addition of the required amount of cross-linker (e.g., adipic dihydrazide or ADH) and carbodiimide (EDAC) as a sterile solution in 90% dimethylformamide (DMF) or 80% isopropyl alcohol (IPA) that has been adjusted to a pH between 2 and 3 using 1 N HCl. As an example, the ADH and EDAC concentrations are brought to 10 mg/ml of final solution. For thoroughness and consistency, each mixing step was performed for 10 minutes. If buffers are not present, the pH will be slightly basic after addition of reagents and prior to acidification. Because the release of genes can be modulated by the degree of cross-linking, this step can be varied. For some applications, the lyophilized matrices are cross-linked for 6 hours. This will enable a relatively rapid release of genes. For sustained gene delivery, the cross-linking step can be performed for 48 hrs. This yields matrices that are more refractory to hydrolysis and gene release. An example of the effects of cross-linking time on the release of DNA is seen in FIG. 9. Similarly, microspheres formulated with DNA and cross-linked for either 6 or 16 hours display altered DNA release kinetics when treated with testicular hyaluronidase. Data demonstrating this property is illustrated in FIG. 6.

Because the components in the unpolymerized mixture are in true solution prior to lyophilization, dispensing metered amounts into the molds allows measured doses to be made.

In a sterile hood, the matrices may be extracted with alcohol while still within the molds by introducing the alcohol to the mold, and allowing the matrix to “soak” in the alcohol solution for the required amount of time. The alcohol is removed by aspiration and the process is repeated 4 times. After extraction of reagents as depicted in the flow chart, the matrices are removed from the molds. There are several methods to accomplish this, but for small scale production, removal with flat bladed forceps is preferred.

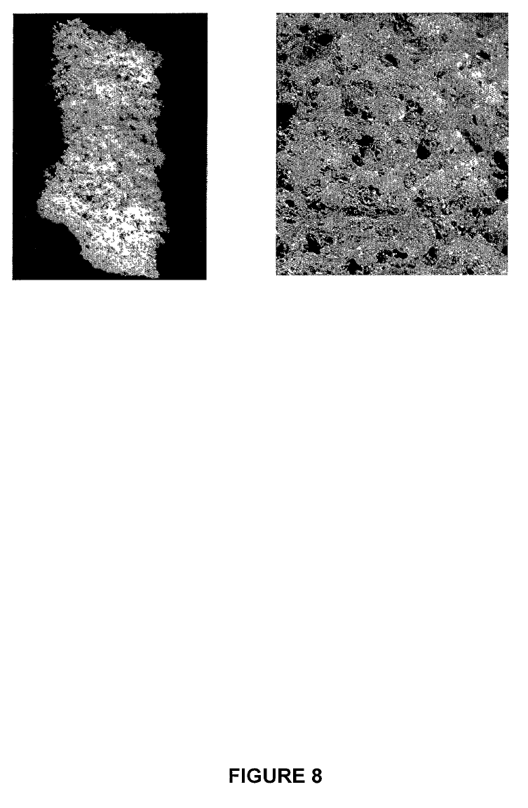

The extracted matrix may be packaged as other ophthalmic products; for example, in a sterile, sealed, protective envelope that controls moisture access within a hard protective case that prevents physical damage to the product. The entire process up to and including placement within a moisture control envelope is conducted with sterile implements using sterile solutions and materials. An electron micrograph of a matrix produced by this method is shown in FIG. 8.

### Example 3

Transfer of Green Fluorescent Protein (GFP) Gene to Living Rat Eyes.

The following experiment demonstrates that GFP, in a dihydrazide derivatized hyaluronic acid bioconjugate may be transferred to living rat ocular cells and that the transformed ocular cells may express functional GFP.

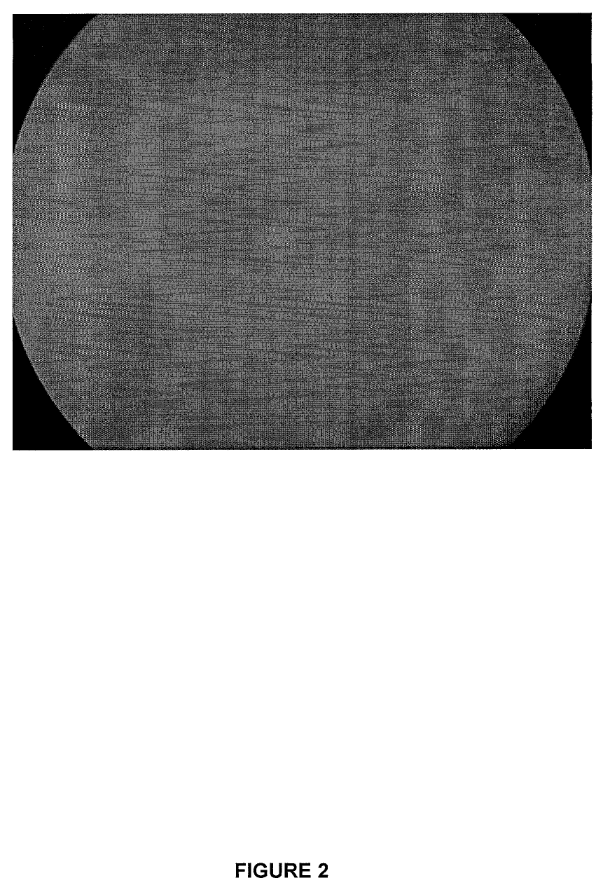

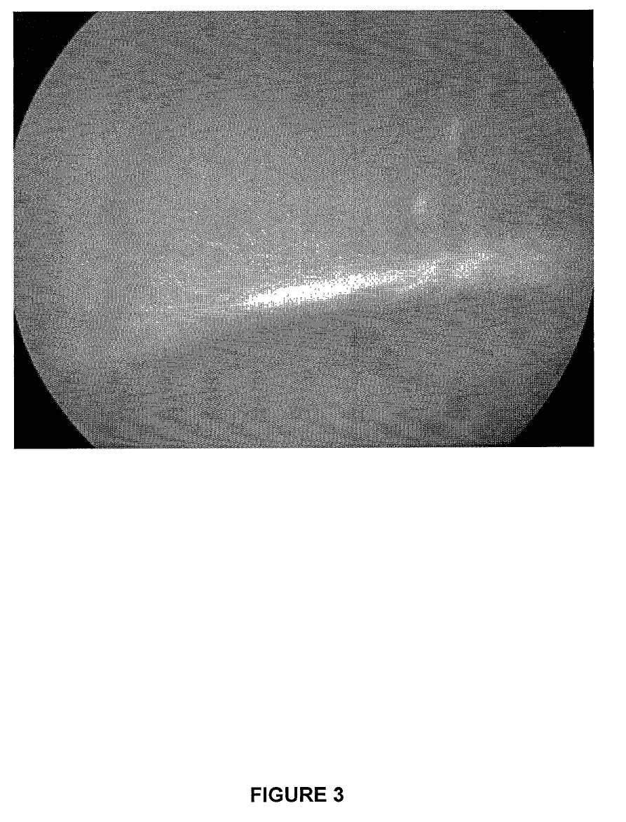

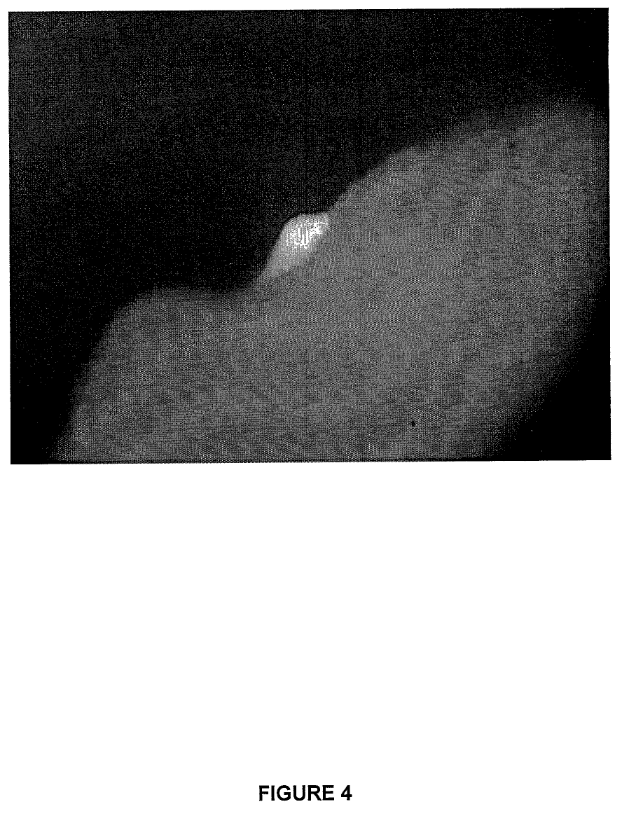

The GFP gene was functionally associated with a promoter which can cause expression of GFP in the rat ocular cells. Nucleic acid comprising the promoter and gene were used to synthesize dihydrazide derivatized hyaluronic acid bioconjugate matrix. A 3 mm×3 mm matrix sample was placed on the corner of a living rat eye for a period of 48 hours. After incubation period, rats were anesthetized with ketamine and the treated eyes were examined microscopically. Under the microscope, the eyes were illuminated with long wave length ultraviolet (U.V.) light to excite the GFP. Micrographs showing GFP fluorescence in the rat eyes are shown in FIGS. 2-4.

The expression of GFP in the rat eyes was also maintained at a high level since the GFP allele used in the experiment had a short half life of only 2-4 hours and yet GFP fluorescence was observed for an extended period after initial transfer of the gene.

### Example 4

Induction of Expression of Hyaluronic Acid in Human Corneal Epithelial Cells

In this example, human corneal epithelial cells are transformed with matrices of the invention comprising mouse HAS1, HAS2 or HAS3. Furthermore, expression of hyaluronic acid in the transformed cells is demonstrated.

Four separate experiments are performed: human corneal epithelial cells are transformed with (1) mouse HAS1 (SEQ ID NO.1), (2) mouse HAS2 (SEQ ID NO.2), (3) mouse HAS 3 (SEQ ID NO.3) or (4) with pcDNA3.1/V5-His A (Invitrogen; Carlsbad, Calif.) plasmid alone. Hyaluronic acid production is then determined for each transformed cell line.

Preparation of matrices. The HAS 1, HAS2 or HAS3 genes are inserted into the plasmid pcDNA3.1/V5-His A (Invitrogen; Carlsbad, Calif.) which drives expression of the HAS 1, HAS2 or HAS 3 genes from a CMV promoter. Matrices comprising each plasmid are formulated using the procedure described above (see Example 2).

Cell line growth. Human corneal epithelial cell line HCE-2 (CRL-11135;ATCC, Rockville, Md.) is grown in MEM medium with 10% Fetal Bovine Serum (FBS) at 37° C. at 5% CO₂.

Transformation. In a 35 mm tissue culture plate, about 3×10⁵ HCE-2 cells are seeded in 2 ml DMEM growth medium. The cells are incubated at 37° C. in 5% CO₂ until the cells are about 50-70% confluent.

Two sterile solutions for each transfection are prepared in 12×75 mm sterile tubes as follows:

Solution A: Dilute DNA/HA matrix bioconjugates in 100 μl serum-free medium ( OPTI-MEM, I Reduced Serum Medium (GIBCO BRL Cat. No. 320-1985)).

Solution B: For each transfection, dilute 2-25 μl of LIPOFECTAMINE Reagent into 100 μl of the serum-free medium.

Solutions A and B are combined and mixed gently, and incubate at room temperature for 15-45 min. Furthermore, the cells are washed once with 2 ml of the serum-free medium. For each transfection, 0.8 ml of serum-free medium is added to each tube containing the cationic lipid-DNA mixture along with porcine testicular hyaluronidase (final concentration: 15 units/ml) and the solution is gently mixed.

Each mixture is poured over the HCE-2 cells and the cells are grown at 37° C. for 48 hours under 5% CO₂.

Hylauronic acid determination. The quantity of hyaluronic acid associated with each cell line is determined using the biotin/streptavidin/horse radish peroxidase conjugated assay discussed below.

After incubation, the transformed cells are fixed and incubated for 1 hour with biotinylated hyaluronic acid binding protein (HABP) followed by washing with MEM and 10% FBS. After washing, the cells are further incubated for 1 hour with streptavidin conjugated horse radish peroxidase and washed with MEM and 10% FBS. The washed cells are then incubated 3,3′,5,5′-tetamethylbenzidine (TMB) and analyzed microscopically.

Results. These experiments demonstrate that the level of hyaluronic acid in the cells of experiments 1-3 (corresponding to HAS1, HAS2 and HAS3 transformed cells, respectively) was measurably greater than that of the cells of experiment 4 (corresponding to cells transformed with pcDNA3.1/V5-His A only).

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

We claim:

1. A method of transfecting a cell of an eye with nucleic acid wherein said nucleic acid comprises a nucleotide sequence which is a member selected from the group consisting of SEQ ID NOs. 1-3, comprising the step of contacting the cell with a dihydrazide derivatized hyaluronic acid/nucleic acid bioconjugate which comprises said nucleic acid.

2. The method of claim 1 wherein the derivatized hyaluronic acid is derivatized with adipic dihydrazide.

3. The method of claim 1 wherein the derivatized hyaluronic acid is derivatized with terephtalate dihydrazide.

4. The method of claim 1 wherein the nucleic acid is a plasmid

5. The method of claim 1 wherein the bioconjugate is in a form which is a member selected from the group consisting of a microsphere, a film, a wafer, a matrix, a hydrogel, a gel and a sol.

6. The method of claim 1 wherein the cell is within an eye of an individual.

7. A method of treating dry eye syndrome in an individual comprising transfecting a cell in an eye of said individual by the method of claim 1.

8. A method for synthesizing a dihydrazide derivatized hyaluronic acid/nucleic acid bioconjugate wherein said nucleic acid comprises a nucleotide sequence which is a member selected from the group consisting of SEQ ID NOs. 1-3;
(a) contacting hyaluronic acid with the nucleic acid to form a mixture;
(b) drying the mixture of hyaluronic acid and the nucleic acid;
(c) suspending the dried mixture in a solution of 90% dimethyl formamide containing a dihydrazide crosslinker and a carbodimide;
(d) adjusting pH of the suspension of step (c) to an acidic range;
(e) isolating the suspended material from the suspension; and
(f) washing the isolated material from step (e) with an alcohol.

9. The method of claim 8 wherein the dihydrazide crosslinker is adipic dihydrazide.

10. The method of claim 8 wherein the dihydrazide crosslinker is terephtalate dihydrazide.

11. The method of claim 8 wherein the carbodiimide is 1-ethyl-3-(3-dimethyl-aminopropyl) carbodiimide.

12. The method of claim 8 which further comprises a step which occurs after step (d) and before step (e) wherein the suspended material is incubated for a period of from about 6 hours to about 24 hours.

13. A bioconjugate produced by the method of claim 8.

14. A method for modulating an extent of crosslinking between a nucleic acid and a dihydrazide derivatized hyaluronic acid in a bioconjugate comprising the method of claim 8 wherein a concentration of the dihydrazide crosslinker of step (c) is increased to increase the extent of crosslinking or decreased to decrease the extent of crosslinking.

15. A method for modulating an extent of crosslinking between a nucleic acid and a dihydrazide derivatized hyaluronic acid in a bioconjugate comprising the method of claim 8 wherein at step (c), the pH is lowered to increase the extent of crosslinking or the pH is raised to decrease the extent of crosslinking

16. An isolated eye cell transfected by the method of claim 1.

17. A dihydrazide derivatized hyaluronic acid/nucleic acid bioconjugate comprising derivatized hyaluronic acid crosslinked to a nucleic acid wherein said nucleic acid comprises a gene which encodes a protein which comprises hylauronan synthase activity and wherein the nucleic acid is a member selected from the group consisting of a nucleic acid comprising a nucleotide sequence which comprises at least 85% sequence identity to the reference nucleotide sequence of SEQ ID NO. 1, a nucleic acid comprising a nucleotide sequence which comprises at least 81% sequence identity to the reference nucleotide sequence of SEQ ID NO. 2, and a nucleic acid comprising a nucleotide sequence which comprises at least 79% sequence identity to the reference nucleotide sequence of SEQ ID NO. 3, wherein identity is determined using a BLASTN algorithm, where parameters of the algorithm are selected to give the largest match between the sequences tested over the entire length of the reference sequence.

18. A dihydrazide derivatized hyaluronic acid/nucleic acid bioconjugate comprising derivatized hyaluronic acid crosslinked to a nucleic acid wherein said nucleic acid comprises a gene which encodes a protein which comprises hylauronan synthase activity and wherein the nucleic acid is a member selected from the group consisting of a nucleic acid comprising a nucleotide sequence which encodes a protein whose amino acid sequence comprises at least 66% sequence homology to the reference amino acid sequence of SEQ ID NO. 4, a nucleic acid comprising a nucleotide sequence which encodes a protein whose amino acid sequence comprises at least 90% sequence homology to the reference amino acid sequence of SEQ ID NO. 5 and a nucleic acid comprising a nucleotide sequence which encodes a protein whose amino acid sequence comprises at least 89% sequence homology to the reference amino acid sequence of SEQ ID NO. 6, wherein identity is determined using a BLASTP algorithm, where parameters of the algorithm are selected to give the largest match between the sequences tested over the entire length of the reference sequence.

19. The bioconjugate of claim 17 wherein the nucleotide sequence of the nucleic acid is a member selected from the group consisting of SEQ ID NOs. 1-3.

20. The bioconjugate of claim 18 wherein the amino acid sequence of the protein is a member selected from the group consisting of SEQ ID NOs. 4-6.

21. The bioconjugate of claim 17 wherein the derivatized hyaluronic acid is derivatized with adipic dihydrazide.

22. The bioconjugate of claim 18 wherein the derivatized hyaluronic acid is derivatized with adipic dihydrazide.

23. The bioconjugate of claim 17 wherein the derivatized hyaluronic acid is derivatized with terephtalate dihydrazide.

24. The bioconjugate of claim 18 wherein the derivatized hyaluronic acid is derivatized with terephtalate dihydrazide.

25. The bioconjugate of claim 17 wherein the nucleic acid is plasmid DNA

26. The bioconjugate of claim 18 wherein the nucleic acid is plasmid DNA

27. The bioconjugate of claim 17 which is in a form which is a member selected from the group consisting of a microsphere, a film, a wafer, a matrix, a gel, a hydrogel and a sol.

28. The bioconjugate of claim 18 which is in a form which is a member selected from the group consisting of a microsphere, a film, a wafer, a matrix, a gel, a hydrogel and a sol.
